(19) United States
(12) Patent Application Publication
(10) Pub. No.: US 2016/0222372 A1
(43) Pub. Date: Aug. 4, 2016

(54) Enzyme/Protein Packaged Bacterial Vesicles for Therapeutic Delivery

(71) Applicant: The Government of the United States of America, as represented by the Secretary of the Navy, Arlington, VA (US)

(72) Inventors: Scott Walper, Springfield, VA (US); Nathan Alves, Fairfax, VA (US); Kendrick Turner, Washington, DC (US); Igor L. Medintz, Springfield, VA (US); George P. Anderson, Bowie, MD (US)

(21) Appl. No.: 15/010,966

(22) Filed: Jan. 29, 2016

Related U.S. Application Data
(60) Provisional application No. 62/241,380, filed on Oct. 14, 2015.
(60) Provisional application No. 62/109,899, filed on Jan. 30, 2015.

(51) Int. Cl. C12N 9/96; C12N 9/16

(52) U.S. Cl. CPC C12N 9/96; C12N 9/16; C12Y 301/08001

(57) ABSTRACT

A method of producing a protein includes providing a bacterial cell expressing both (a) a protein of interest fused to one of the SpyTag/SpyCatcher pair and (b) an outer membrane protein fused to the other of the SpyTag/SpyCatcher pair; causing the bacterial cell to express both of the protein of interest fusion and the outer membrane protein fusion in outer membrane vesicles; and purifying the outer membrane vesicles.

### CROSS-REFERENCE TO RELATED APPLICATIONS

This Application claims the benefit of both U.S. Provisional Application No. 62/109,899 filed on Jan. 30, 2015 and U.S. Provisional Application No. 62/241,380 filed on Oct. 14, 2015, the entirety of each of which is incorporated herein by reference.

### BACKGROUND

Most if not all bacteria studied to date, including both Gram-negative and Gram-positive bacteria, produce outer membrane vesicles (OMVs) from their surface. These small (30-200 nm) unilamellar proteoliposomes serve various functions from cell-cell signaling to packaging of virulence factors in pathogenic bacterial strains to infect host cells. Various studies have been performed demonstrating use of bacterial OMVs for packaging material and facilitating delivery of proteins of interest. Due to the diverse circumstances for which OMVs are formed and the complex composition of both their packaged contents and their lipid-protein shell a discrete pathway for the packaging of cellular components has not yet been elucidated.

Large-scale production of recombinant proteins, peptides, and small molecules (heretofore referred to as recombinant products) using a bacterial host is a well-established process that has led to significant successes in the manufacture of industrially relevant enzymes, diagnostic reagents, and therapeutics. Historically, individual recombinant products have been produced in a single microbial culture, purified using a broad range of techniques, then stored for eventual use. These established methods have been successful but can prove limiting if multiple recombinant products are required to facilitate a specific process as each component is produced separately and stored for eventual use. In addition to limitations due to the complexity associated with the manufacture of the individual components of a multi-component system, many recombinant products (particularly, but not limited to, enzymes) show a propensity to lose activity over time if improperly stored, subjected to conditions of temperature variations, freezing and thawing, or instability to lyophilization. Furthermore, in some instances, the accumulation of recombinant products within the host microorganism can lead to a reduction in culture viability due to inhibition of cellular processes or even cellular toxicity which in many instances limits the yield of recombinant product that can be attained given a fixed reactor volume.

### BRIEF SUMMARY

Packaging recombinant products within OMVs and secreting them from the bacteria reduces intracellular concentrations of the product and thus alleviates toxicity, increases culture viability, and in turn increases the overall yield of recombinant product.

In one embodiment, a method of producing a protein includes providing a bacterial cell expressing both (a) a protein of interest fused to one of the SpyTag/SpyCatcher pair and (b) an outer membrane protein fused to the other of the SpyTag/SpyCatcher pair; causing the bacterial cell to express both of the protein of interest fusion and the outer membrane protein fusion in outer membrane vesicles; and purifying the outer membrane vesicles.

### BRIEF DESCRIPTION OF THE DRAWINGS

FIG. 1 shows crystal structures for the proteins utilized in the biorthogonal membrane conjugation of phosphotriesterase (PTE) for packaging into outer membrane vesicles: OmpA, PTE, SpyTag, and SpyCatcher, found under Protein Data Bank (PDB) 2GE4, 1PTA, 4MLI, 4MLI, respectively. The schematic represents the N-terminal OmpA-SpyTag and PTE-SpyCatcher forming an isopeptide bond at the outer membrane surface of the bacteria. This membrane fusion facilitates incorporation of the PTE within the OMVs that are released from the bacteria surface due to the directional insertion of OmpA into the bacterial membrane.

FIG. 2A shows the weight of cell pellets for each construct (defined as N-, Internal-, or C-terminal OmpA-ST fusion; Arabinose; IPTG added to the culture) as indicators of general culture viability, demonstrating no significant difference in toxicity exhibited by any of the mutants when compared to nonmutant BL21(DE3) E. coli. FIG. 2B shows OMV production for each construct was quantified via NanoSight particle tracking demonstrating a significant increase in OMV production present in the CA, NAI, and CAI cultures (55-fold dilute). OMVs were also quantified in the ultracentrifugation (UC) supernatant (Sup.) demonstrating a nearly complete recovery of OMVs in the UC pellet. All data represents means (±SD) of triplicate experiments. See “Production and Purification of OMVs” section for definitions of cultures.

FIG. 3A shows a scanning electron micrograph (SEM) images of purified OMVs from native E. coli. Representative NanoSight size distributions and total vesicle concentration averaged over 90 s sample reads for 55-fold dilute. FIGS. 3B-3D show (FIG. 3B) native E. coli, PTE-SC in the absence of arabinose (PTE-No A), and in the presence of arabinose (PTE-A); (FIG. 3C) N-terminal OmpA-ST with PTE-SC in the presence of arabinose (NA), IA, CA; (FIG. 3D) N-terminal OmpA-ST with PTE-SC in the presence of arabinose and IPTG (NAI), IAI, CAI.

FIGS. 4A and 4B show (FIG. 4A) SDS-PAGE and (FIG. 4B) Western blot of the purified OMV UC pellets of all of the various constructs demonstrating relative abundance of OmpA-ST, native OmpA, PTE-SC, and the OmpA-ST/PTE-SC fusion. A His-tag was included in the mutant forms of OmpA and PTE to facilitate visualization via an anti6× His antibody. PTE is known to dimerize, and this is evident on the blot by the presence of larger molecular weight His-tagged species.

FIGS. 5A-5D show (FIG. 5A) PTE activity comparison between the E. coli cell pellets, UC OMV pellets, and UC supernatant demonstrating the total amount of PTE produced and general OMV packaging efficiency via an initial velocity determination utilizing paraoxon as a chromogenic substrate. (FIG. 5B) Triton X-100 (0.5%) was utilized to disrupt the OMV bilayer to allow unimpeded access of paraoxon to the OMV interior. Very little activity difference was observed in the presence or absence of T100 indicating that paraoxon passes through the pores on the OMV. (FIG. 5C) Comparison of the percent PTE trapped in the cells, exported into the culture media, or packaged in OMVs. (FIG. 5D) Normalized PTE activity per OMV demonstrated improved PTE packing efficiency in the CA construct.

FIG. 6A shows PTE kinetic data fit to the standard Michaelis-Menten enzyme kinetics equation for NAI and CAI UC pellet, CAI and CA UC supernatant. FIG. 6B is a Lineweaver-Burk analysis used for determining K_{M} and k_{cat}/K_{M} (48, 4.4×10⁷; 50, 4.4×10⁷; 44, 4.9×10⁷; 103 μM, 2.3×10⁷s⁻¹M⁻¹, respectively) demonstrating consistent PTE enzyme kinetic parameters as free enzyme (90 μM, 2.7×10⁷ s⁻¹ M⁻¹) with R²>0.999 in all cases.

FIG. 7 shows results of a freeze-thaw stability test of PTE-SC packaged within NAI and CAI OMVs compared to free PTE-SC purified from the UC supernatant of the CAI construct. Four cycles of freeze-thaw between −80° C. and room temperature were carried out, and the percent PTE activity was directly compared via initial velocity measurements utilizing paraoxon as a substrate. Packaged PTE-SC in both the NAI and CAI constructs exhibited heightened resistance to inactivation compared to free PTE-SC.

### DETAILED DESCRIPTION

Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used in this specification and the appended claims, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

Overview

Described herein are techniques for packaging recombinant products within bacterial outer membrane vesicles (OMVs) for secretion from bacteria and subsequent purification. Potential applications include the packaging of enzymes OMVs to perform simple (single enzyme) or multistep (two or more enzymes) controlled enzymatic reactions. The technology described encompasses the directed packaging of recombinantly-manufactured proteins, peptides, or small molecules into the forming outer membrane vesicles of the host bacterium for the purpose of conducting a process outside the confines of the bacterium (i.e. in the extracellular space outside of the cytoplasmic or periplasmic compartment). This includes, but is not limited to, the development of biological sensors (for example, with detection protein and reporter simultaneously packaged), biological nanobioreactors (multistep pathways for synthesis or degradation pathways), or the formation of nanoparticles within the vesicle using sequestered biomineralization peptides.

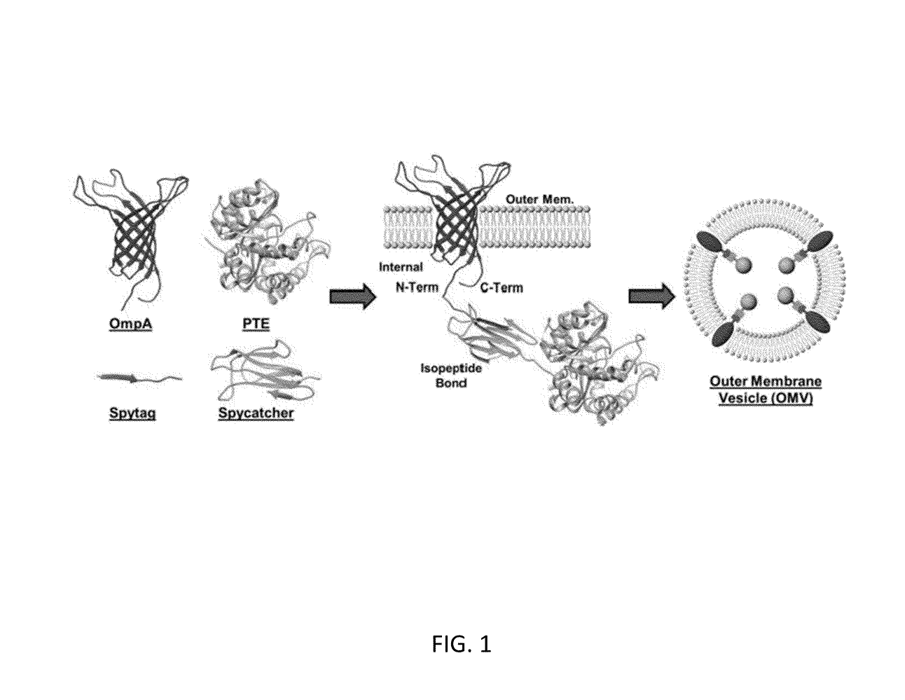

This technique addresses several potential complications encountered in the microbial manufacture of recombinant products such as enzymes, peptides, and small molecules while also improving the stabilization and storage of these products once purified from the cell culture. As shown in the exemplary scheme depicted in FIG. 1, bacterial outer membrane vesicles (OMVs) are used a vehicle to export a protein of interest (the example uses the enzyme phosphotriesterase, which could easily be substituted with other recombinant proteins, peptides, and small molecules) from a microbial cell culture during the production process. Removing recombinant products from the microbe as it is being manufactured through OMV packaging has several advantages to the aforementioned microbial production processes including improving culture viability and offering a mechanism of storage that has been shown to improve the stability of the recombinant product over a range of conditions (detailed below).

Although the example used the membrane protein OmpA, it is expected that other bacterial membrane proteins could be substituted. Bacterial membrane proteins of interest for modification include but are not limited to: OmpA, OmpC, OmpF, PorA, PorB, OprE, OprF, FimD, FccA, FhuE, FepA, FhuA, YddB, SLT, MalM, PRC, FkpA, OppA, FimA, GInH, HdeA, LivJ, MltA, MltB, MltE, OmpW, OmpX, BtuB, OmpT, MexA, MexE, MtrE, TolC, OstA and other porin, transmembrane, membrane anchored proteins.

Similar, the SpyTag/Spycatcher bioorthogonal linkage might be replaced with alternative conjugation strategies that include split proteins, split inteins, and coiled coils, just to name a few. Also, other methods of membrane anchoring of the recombinant product itself could be employed such as addition of a lipoylation sequence, the addition of a hydrophobic domain, or transmembrane motif to facilitate immobilization of the recombinant product within the outer membrane directly. While membrane anchoring is utilized here to drive increased packaging of the recombinant product into the OMVs, the membrane anchoring may not be necessary to achieve increased stability of the packaged recombinant product.

The below example purified OMVs by filtration and ultracentrifugation, however other techniques can be used either in the alternative or in conjunction with one or both of these processes. It is also possible to use purification, for example using a polyhisitidine tag. Other tags of interest for inclusion within OMVs include but are not limited to: avitag, calmodulin-tag, polyglutamate-tag, E-tag, FLAG-tag, HA-tag, His-tag, Myc-tag, S-tag, SBP-tag, Softag 1, Softag 3, Strep-tag, TC-tag, V5-tag, VSV-tag, Xpress-tag, Isopeptag, Spytag, BCCP, Glutathione-S-transferase-tag, Maltose binding protein-tag, Green fluorescent protein-tag, Nus-tag, Thioredoxin-tag, Fc-tag and other peptide, small molecule, protein, cleavable and non-cleavable based tags.

In various embodiments, OMVs are subject to quality control methods to ensure removal of bacterial DNA and contaminating proteins prior to use.

The “plug-and-play” characteristics of controlled packaging of enzymes and other proteins in OMVs may lead to not only the development of novel therapeutics, but also bacterial derived reagents for use in a broad range of applications such as remediation, commercial processing, and others. Complex processes such as solid waste disposal, biomass degradation, and chemical warfare remediation frequently require extensive microbial communities working in concert to process a material from its starting form to something useful or less dangerous. In many instances such communities require optimal growth conditions which are not easily maintained or even impossible to mimic. Combining the tools of synthetic biology and directed bacterial OMV packaging it will be possible to simultaneous encapsulate all enzymatic processes to perform the task into a single nanobioreactor. Enzyme-filled OMVs will then serve directly as reagents eliminating the need to release the original bacteria used to produce the OMVs. This is a common practice that has been exploited in commercial products such as laundry detergent and dishwashing pods.

Simultaneous packaging of multiple enzymes from a single pathway allows for the development of novel reagents for the remediation of environmental contaminants using standard laboratory strains. With established molecular manipulation protocols and reduced pathogenicity/toxicity, laboratory strains offer a rapid avenue of construction of OMV-based reagents for such applications as chemical contamination remediation. Typically, organophosphate contamination of soil and water reservoirs can result from excessive pesticide usage in agricultural industries, however, many chemical warfare agents are molecularly very similar to this toxic reagent. Microbes such as Bacillus and Pseudomonas species have developed enzymatic pathways capable of reducing a variety of these toxic compounds to inert bioproducts.

A brief list of representative bacteria that are of interest include but are not limited to: (likely suited to therapeutic applications) Lactobacillus species, Bifidobacterium species, Salmonella enterica, Heliobacter pylori, Escherichia coli; and (environmental applications) Bacillus coagulans, Pseudomonas aeruginosa, Pseudomonas diminuta, Bacillus subtilis, Bacillus thuringiensis, Escherichia coli.

### EXAMPLE

Introductory Remarks

Further details regarding this work can be found in ACS Appl. Mater Interfaces, 2015, 7 (44), pp 24963-24972 and the accompanying Supporting Information, incorporated herein by reference.

To help drive packaging of the phosphotriesterase enzyme into the vesicles, a synthetic linkage was sought between the enzyme and a known protein present in the outer membrane at high abundance. There are various synthetic strategies for pairing two different proteins within a biological system that include the following: split proteins, coiled coils, and split inteins, just to list a few. For the purposes of this application the SpyCatcher/SpyTag bioconjugation system was selected which employs a fibronectin-binding protein (FbaB) from Streptococcus pyogenes which is a split protein that employs two subunit domains referred to as the SpyCatcher (SC) (SEQ ID No: 9) and SpyTag (ST) (SEQ ID No: 6) domains. Unlike many split protein systems, the SC/ST system provides for the formation of an isopeptide bond between proximal aspartic acid and lysine amino acid residues. This interaction and bond formation happens spontaneously as it does not require the addition of chaperone proteins, catalytic enzymes, or cofactors. The reaction occurs at room temperature and over a wide range of physiologically relevant conditions.

OmpA serves as a membrane tethering protein since it is a highly expressed porin protein present in the bacterial outer membrane and subsequent OMVs. Native OmpA is a 37.2 kDa transmembrane porin protein implicated in the transport of small molecules <2 nm in size across the bacterial membrane. OmpA can effectively be split into two separate structural domains, a transmembrane β barrel motif and a periplasmic soluble C-terminal portion known to interact with the peptidoglycan. While historical studies have shown that deletion of OmpA is nonlethal, this study maintained the genomic OmpA in addition to the recombinantly expressed OmpA-ST construct. This allowed the determination of whether or not increased production of the membrane protein leads to decreases in cell viability and membrane destabilization and determine its effect on the overall OMV production.

Phosphotriesterase (PTE) (EC 3.1.8.1) from Brevundimonas diminuta, containing a binuclear Zn/Zn active site, was selected as the enzyme to be packaged within the OMVs. PTE has the ability to break down organophosphates through a hydrolysis reaction converting aryldialkylphosphates into less toxic dialkylphosphates and aryl alcohols. Organophosphate exposure is extremely dangerous as it impairs proper neurotransmitter function through inhibiting the hydrolysis of acetylcholine by acetylcholinesterase at neuromuscular junctions. Significant exposure to organophosphates most commonly causes uncontrollable convulsions and typically results in death via asphyxiation. PTE is a highly promiscuous enzyme capable of hydrolyzing a broad range of pesticides as well as V and G type nerve agents. A high catalytic activity is observed for the substrate paraoxon, a pesticide analog of chemical warfare agents such as sarin and VX gas. The use of PTE in this representative system of organophosphate degradation provides for an excellent model with applications for the environmental remediation of organophosphate contaminated regions. Remediation is a necessary step to prevent continued organophosphate exposure in contaminated regions which would otherwise be rendered uninhabitable for extended periods of time.

PTE exhibits a very high enzymatic activity and has also previously been expressed in Escherichia coli making it an excellent model enzyme for assessing packaging efficiency into bacterial OMVs. In order to take advantage of the SC/ST coupling system a ST domain was fused to OmpA (OmpA-ST), and a SC domain was fused to the C-terminal portion of PTE (PTE-SC, FIG. 1).(20, 30, 31) By fusing each subunit of the split protein SC/ST to the OmpA and PTE the two proteins will be brought together in vivo which will drive packaging of the PTE into the OMVs. While PTE was selected for this unique application, the results of this study can be used to design analogous protein packaging strategies for use in diverse pharmaceutical delivery, medical diagnostic, and environmental remediation applications.

FIG. 1 shows crystal structures for the proteins utilized in the biorthogonal membrane conjugation of phosphotriesterase (PTE) for packaging into outer membrane vesicles: OmpA, PTE, SpyTag, and SpyCatcher, found under Protein Data Bank (PDB) 2GE4, 1PTA, 4MLI, 4MLI, respectively. Three separate OmpA-SpyTag fusion constructs were synthesized: N-terminal (N) (SEQ ID No: 1), an internal (I) OmpA loop fusion (SEQ ID No: 2), and C-terminal (SEQ ID No: 3). The figures provides a schematic representation of N-terminal OmpA-SpyTag and PTE-SpyCatcher forming an isopeptide bond at the outer membrane surface of the bacteria. This membrane fusion facilitates incorporation of the PTE within the OMVs that are released from the bacteria surface due to the directional insertion of OmpA into the bacterial membrane.

Bioorthogonal Sites for Conjugation

To ensure the optimal ST-SC interaction was attained, OmpA-ST fusions were constructed placing the ST at three periplasmically facing positions; either the N-termini (N), within a random coil loop (I), or at the C-termini (C), with the resulting proteins having SEQ ID Nos. 1, 2, and 3, respectively. The location of the ST (SEQ ID No. 6) will have an effect on the conjugation efficiency of the ST-SC interaction and must be experimentally determined. The recombinant OmpA was further modified by removing the native C-terminal portion of the protein, which is known to interact with the peptidoglycan. The peptidoglycan, which is a dense network of cross-linked sugars and amino acid residues, provides a relatively rigid framework to stabilize the outer membrane through interactions between transmembrane proteins present in the outer membrane that also bind to the peptidoglycan, such as OmpA. By removing the C-terminal portion of OmpA it is anticipated that the outer membrane may destabilize since there will be less trans-periplasmic interactions between the outer membrane and the peptidoglycan. Maintaining the genomic OmpA prevents this destabilization effect from having a significant impact on the bacterial viability. The C-terminal deletion of OmpA, in addition to the ST-SC interaction, helps to promote vesiculation and in turn facilitate packaging of the PTE-SC within the OmpA-ST decorated vesicles. The OmpA fusions also had N-terminal leader sequences (SEQ ID No: 5).

Design of the Expression Plasmids

Genes encoding for a truncated OmpA with the SpyTag sequence appended to the either the N-terminus, C-terminus, or an internal loop were synthesized by GenScript (Piscataway, N.J.) in a pUC57 shuttle vector with flanking NcoI and NotI restriction sites. The truncated OmpA consisted of native OmpA with the unessential C-terminal domain portion deleted. The spy tag in each construct was flanked by a spacer amino acid sequence (GGGS). The SpyTag insertion site at the internal loop was chosen based on the published tolerance for insertion at this location. Synthesized plasmids were digested with NcoI-HF and NotI-HF (New England Biolabs, Ipswich, Mass.) and cloned into identical sites in the pET22b expression vector (Novagen, Billerica, Mass.).

A second expression vector utilizing a compatible origin of replication was constructed for the coexpression of the PTE-SC construct (with resulting protein having SEQ ID No: 4). The pACYC184 vector (New England Biolabs) which contains a p15a origin served as the backbone for this construct. The regulatory elements for arabinose induction were amplified via PCR from the pBAD/Myc-His plasmid (Life Technologies) using primers that also encoded the twin-arginine translocation substrate TMAO reductase (TorA, SEQ ID No: 8), a hexahistidine sequence, and several unique restriction endonuclease cleavage sites (vector referred to as pACYC184 AraC). The phosphotriesterase and SpyCatcher genes were combined through a series of PCR amplification, restriction digest reactions, and ligations. The SpyCatcher gene (with the protein having SEQ ID No: 9) was amplified from a bacterial expression vector using primers that generated flanking XhoI sites and a 5′-Acc65I site just upstream of the SpyCatcher sequence. The PCR product was cloned to the pMinit PCR cloning vector (New England Biolabs) which served as the shuttle vector for cloning of the PTE gene. PTE (with the resulting protein having SEQ ID No: 10) was amplified separately using primers to generate flanking Acc65I sites and a short amino acid spacer sequence. The PCR product was digested and cloned to the pMinit SpyCatcher construct whose sequence was confirmed. Both the pMinit PTE-SC and pACYC184 AraC were digested with XhoI and gel purified, and the relevant fragments were ligated using T4 DNA ligase.

Production and Purification of OMVs

The E. coli strain BL21(DE3) was cotransformed with both OmpA-ST and PTE-SC plasmid constructs and maintained on solid medium or in liquid culture in the presence of ampicillin (50 μg/mL) and chloramphenicol (25 μg/mL). For OMV production a 5 mL overnight served as a starter culture to inoculate 50 mL baffled culture flasks which were allowed to expand for 3 h at 37° C. until an OD of 0.6-0.8 was reached. Where indicated, arabinose was added at a 0.2% final concentration initiating production of the PTE-SC. After an additional 3 h incubation period, IPTG was added to a final concentration of 0.5 mM to boost OmpA-ST production, and the culture was allowed to grow for an additional 18 h. Individual 50 mL cultures were inoculated for each of the three (N, I, C) OmpA-ST fusions with PTE-SC as well as PTE-SC by itself. Separate cultures for each were compared in the presence of only arabinose, “A” (NA, IA, CA, PTE-A), and in the presence of both arabinose and IPTG, “AI” (NAT, IAI, CAI). All samples were compared to nonmutant control BL21(DE3) cultures for cell pellet weight and OMV production levels.

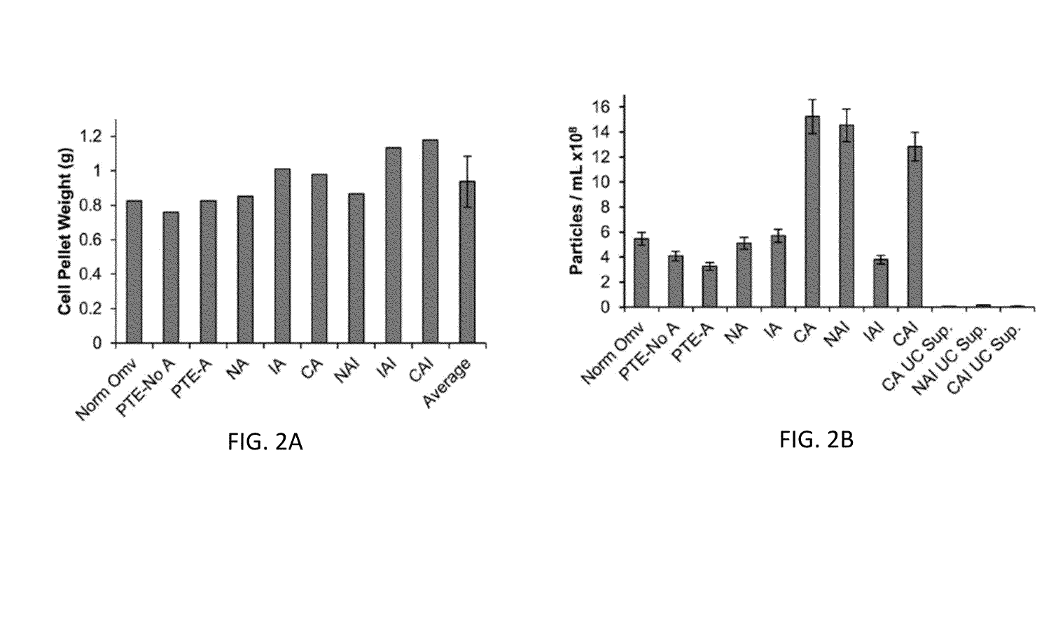

At the completion of the growth phase, the intact cells and larger cellular components were removed from the culture media via centrifugation and 0.45 μm membrane filtration. Cell pellets were weighed to assess general culture viability and to verify that none of the constructs were toxic to the cells (FIG. 2A). OMVs were then pelleted at ˜150,000 g using an ultracentrifuge for 3 h at 4° C. The OMV depleted culture media was decanted, and the OMV pellet was resuspended in PBS pH 7.4. This method of OMV purification was selected to ensure that all OMVs were captured from the culture media to allow for accurate quantitation of OMV production as well as PTE packaging. Many techniques, such as density gradient fractionation, are utilized to purify OMVs and can provide for highly pure products; but the additional selection step often biases results as the OMV size distribution can be fairly broad (30-200 nm diameters), and their composition can impact their density and subsequent retrieval. Controls were carried out to ensure that the OMVs were fully depleted from the culture media at the conclusion of ultracentrifugation. It was also verified that free PTE-SC secreted into the culture media, which was observed in a few of the constructs, did not associate with the external surface of the OMVs and that the centrifugal force necessary to pellet the OMVs was not sufficient to pellet non-OMV encapsulated PTE-SC. The expression vector of PTE-SC contained a periplasmic localization tag, and it was not expected to see free PTE-SC released into the culture media at any appreciable quantities. The release of free PTE-SC was likely a result of membrane destabilization caused by the very high overexpression of the OmpA-ST. Samples of each fraction were collected and were then utilized in all subsequent analysis.

OMV Characterization

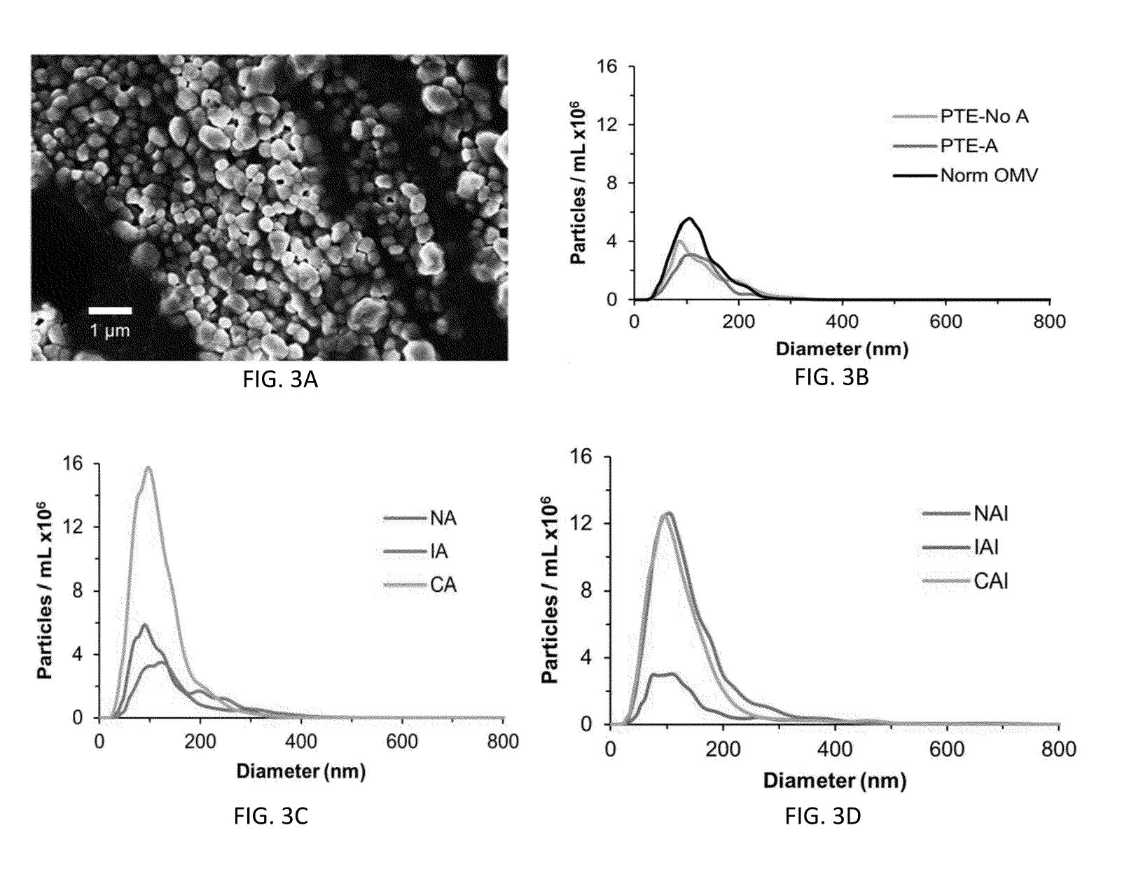

The OMV collected through ultracentrifugation (UC) from each construct were assessed for overall production level and size distribution utilizing a NanoSight LM10 particle tracking system. UC concentrated vesicles were diluted 2,000-fold in PBS, and particle tracking was carried out at room temperature via analysis of 90 s video clips. OMV production levels remained unchanged across many of the constructs, when compared to normal OMV production (5.47×108 particles/mL), with the exception of increased OMV production levels with CA, NAI, and CAI constructs exhibiting 15.3, 14.6, and 12.8×108 particles/mL, respectively (FIG. 2B). Analysis of the UC supernatants verified nearly a complete depletion of OMVs from the culture media. Despite the different levels of OMV production exhibited by each construct, they all demonstrated a very similar size distribution with an average hydrodynamic diameter of 136±67 nm (FIGS. 3B-D). The UC purified native OMVs were also characterized using SEM to visualize vesicle morphology and relative purity (FIG. 3A).

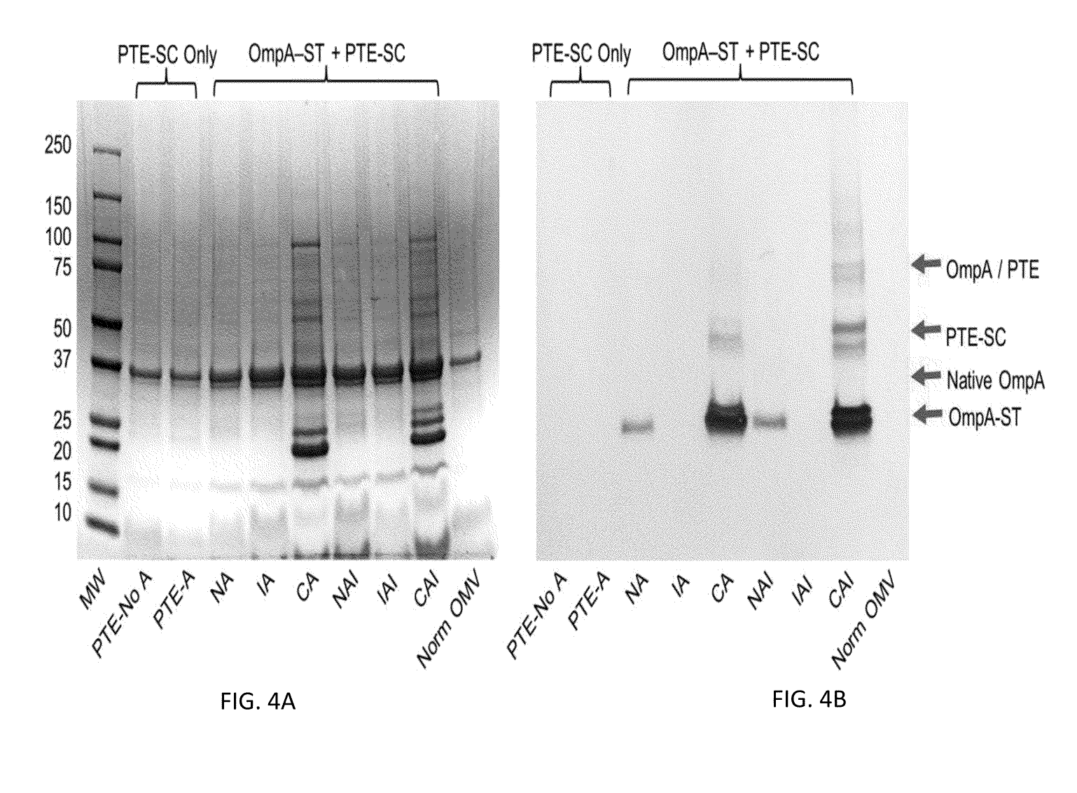

The total protein content of the purified OMVs was analyzed via SDS-PAGE demonstrating relative protein production levels (FIG. 4A). In all OmpA-ST samples there was a marked increase in the level of native OmpA, OmpC, and OmpF (˜35 kDa) production. Present in only the CA and CAI samples were bands that represent the mutant OmpA-ST (calculated molecular weight of 23 kDa). Two OmpA-ST bands are present in these samples exhibiting a small difference in apparent MW which is likely the result of overexpression of OmpA-ST overwhelming the E. coli machinery resulting in improper cleavage of the short peptide leader sequence which adds 2.2 kDa to the MW of the protein construct. Also present in only the CA and CAI samples are bands that represent expression of PTE-SC (calculated molecular weight of 51 kDa) and the OmpA-ST/PTE-SC isopeptide ST-SC fusion product (calculated molecular weight of 74 kDa).

The samples were also analyzed by Western blot utilizing an anti-6×His antibody to probe for each of the mutant proteins. The dominant expression of OmpA-ST was observed in the CA and CAI samples, but OmpA-ST can also be seen at relatively low levels in the NA and NAI samples (FIG. 4B). No expression was observed in native OMV, PTE-SC only, IA and IAI samples. Appreciable ST-SC fusion can be seen in the CAI sample, and to a lesser degree in the CA sample, as indicated by multiple higher molecular weight bands present on the blot. It is important to note that despite high levels of OmpA-ST present there is not a complete conversion of the free PTE-SC to the ST-SC isopeptide fusion. This can be in part due to the microenvironment not being ideal for isopeptide bond formation or a result of steric hindrance at the membrane surface due to other transmembrane and membrane bound proteins which do not inhibit noncovalent association of the ST and SC but prevent the necessary degrees of freedom to facilitate isopeptide bond formation

PTE Expression Levels

Each construct was split into three primary fractions, the cell pellet to assess general PTE-SC production, the UC supernatant to assess the freely exported PTE-SC, and the UC purified OMVs to assess PTE-SC packaging efficiency. A paraoxon activity assay was utilized to determine the amount of active PTE-SC present in each fraction as determined by the initial velocities at a fixed substrate concentration. PTE-SC, when expressed on its own, in the presence of arabinose, resulted in a relatively low level of overall production with very little free PTE-SC or OMV packaged PTE-SC observed. In the absence of arabinose there was almost no production of PTE-SC demonstrating relatively tight regulation utilizing the pACYC184 expression vector. No endogenous PTE activity was observed in the normal E. coli samples tested.

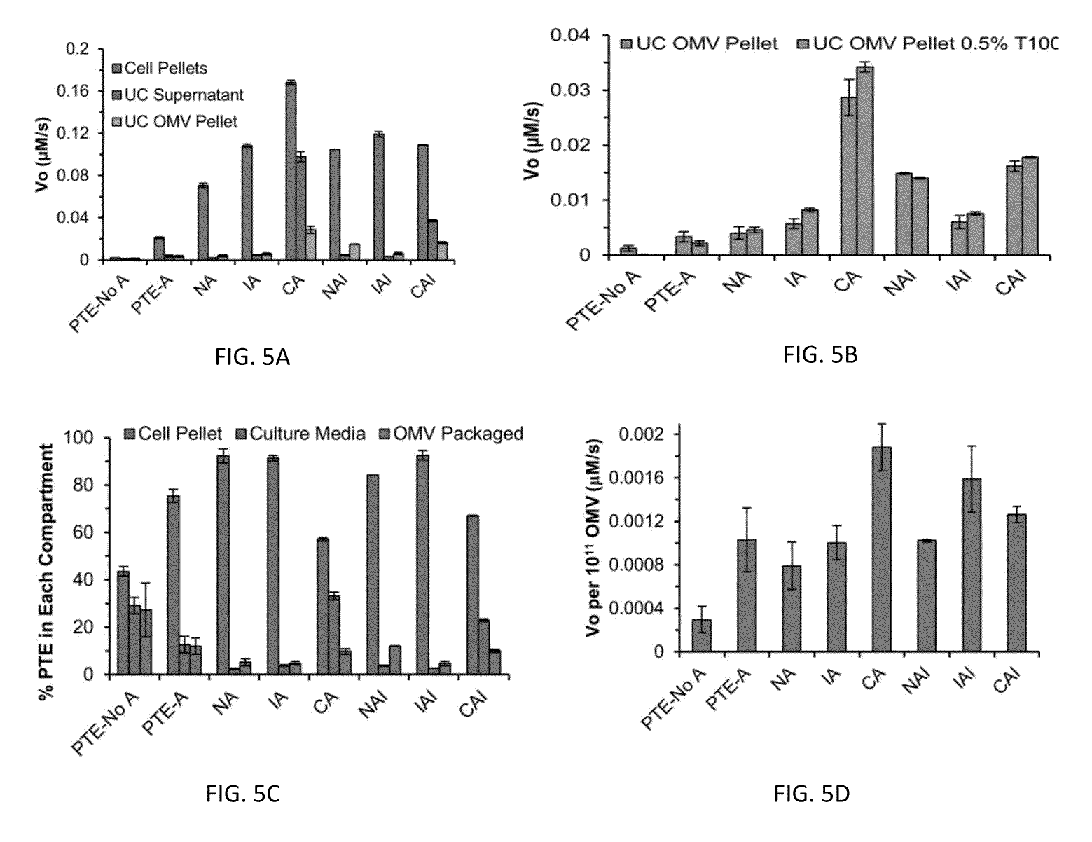

In all instances, the coexpression of PTE-SC with OmpA-ST resulted in a marked increase in the overall production of PTE-SC, exhibiting a minimum increase in PTE-SC production of 3.4-fold (FIG. 5A). As seen here, expression of recombinant PTE by itself exhibited very low protein yields likely due to a loss of viability as a result of toxicity following induction of the PTE. This is in contrast to the observations of this study in which we observed high levels of PTE production in several of the cotransfected cultures and little to no reduction in culture viability. While the primary increase in PTE-SC was observed in the cell pellets, both samples CA and CAI exhibited a relatively large amount of free PTE-SC secreted into the culture media. These samples also exhibited hypervesiculation which may have resulted in a leakier outer membrane causing more free PTE-SC to escape from the periplasm. Overall, the highest level of PTE-SC expression was observed in the CA sample demonstrating a nearly 2-fold increase in PTE-SC production compared to the next highest expression sample of CAI and a 23.6-fold increase over PTE-SC expressed in the absence of OmpA-ST

PTE Packaging

To determine the amount of PTE-SC that was encapsulated within the OMV fraction, the paraoxon enzyme assays were carried out in two different N-Cyclohexyl-2-aminoethanesulfonic acid (CHES) buffers (pH 8.0) with or without the addition of 0.5% Triton X-100. Triton was added to the sample buffer to help facilitate rupturing of the vesicle to allow the PTE-SC access to the circulating paraoxon. A range of Triton values was tested from 0 to 5%, and vesicle rupture was verified via NanoSight analysis. PTE activity was also assessed over the range of Triton values, and 0.5% Triton was selected based on its ability to rupture the vesicles while having minimal impact on PTE activity. Interestingly, the PTE-SC activity for the OMV packaged PTE-SC was largely unaffected by the addition of the Triton indicating that paraoxon relatively freely enters the vesicles (FIG. 5B). This result was unexpected but not surprising since the vesicles are decorated with various porin proteins whose purpose is to shuttle small molecules across the membrane and into the bacteria. Paraoxon, having a molecular weight of only 275.19 Da, is sufficiently small to utilize these porin proteins. Based on this result no further assays were carried out in the presence of Triton. This phenomena is likely not broadly applicable to other enzyme/substrate systems and would have to be experimentally verified for each unique application.

Confident that the assay accurately quantifies the activity of OMV packaged PTE-SC, the constructs were then compared. As suspected, based on the SDS-PAGE and Western blot results, the most PTE-SC packaging was observed in the CA sample, with CAI and NAI both coming in second with nearly half the PTE activity of CA (FIG. 5B). CA, CAI, and NAI packaged 9.7, 10.0, and 12.0% of the total amount of PTE-SC each construct produced, respectively (FIG. 5C) Taking this into consideration the NAI construct produced a significant amount of PTE-SC and packaged the highest percent of the PTE-SC into vesicles without secreting a large portion of free PTE-SC demonstrating the tightest control over packaging of PTE-SC. Comparing the NA and NAI constructs demonstrates that the increase in production of the mutant OmpA-ST, through IPTG activation, results in a large increase in vesiculation as well as increasing the amount of packaged PTE-SC. Other constructs, such as PTE-SC in the absence and presence of arabinose, demonstrated an apparent high efficiency of packaging PTE-SC into the vesicles but produced very little PTE-SC overall, reducing their utility.

Up until this point the various constructs were directly compared, at the physiological production levels unique to each construct, for overall PTE-SC production and the relative distribution of PTE-SC activity within the cell pellet, free in solution, and packaged within the OMVs. To assess the true packaging efficiency for each construct, the PTE-SC activity in the OMV fraction should be normalized to the number of OMVs produced by each construct. This allows for a direct comparison of relative PTE-SC activity per vesicle. Despite a wide range of PTE-SC levels across all of the constructs, the packaging efficiency remained nearly unchanged across most of the samples: PTE-A, NA, IA, NAI, and CAI being on the high end of average (FIG. 5D). The CA construct exhibited the highest packaging efficiency at ˜2-fold the endogenous packaging observed in the PTE-A sample. While the IAI construct also exhibited improved PTE-SC packaging efficiency at 1.6-fold above endogenous levels it produced 4.7-fold less PTE-SC in the OMV fraction with 4.0-fold lower overall vesicle production compared to the CA construct rendering the IAI construct much less useful.

Packaged PTE Enzyme Kinetics

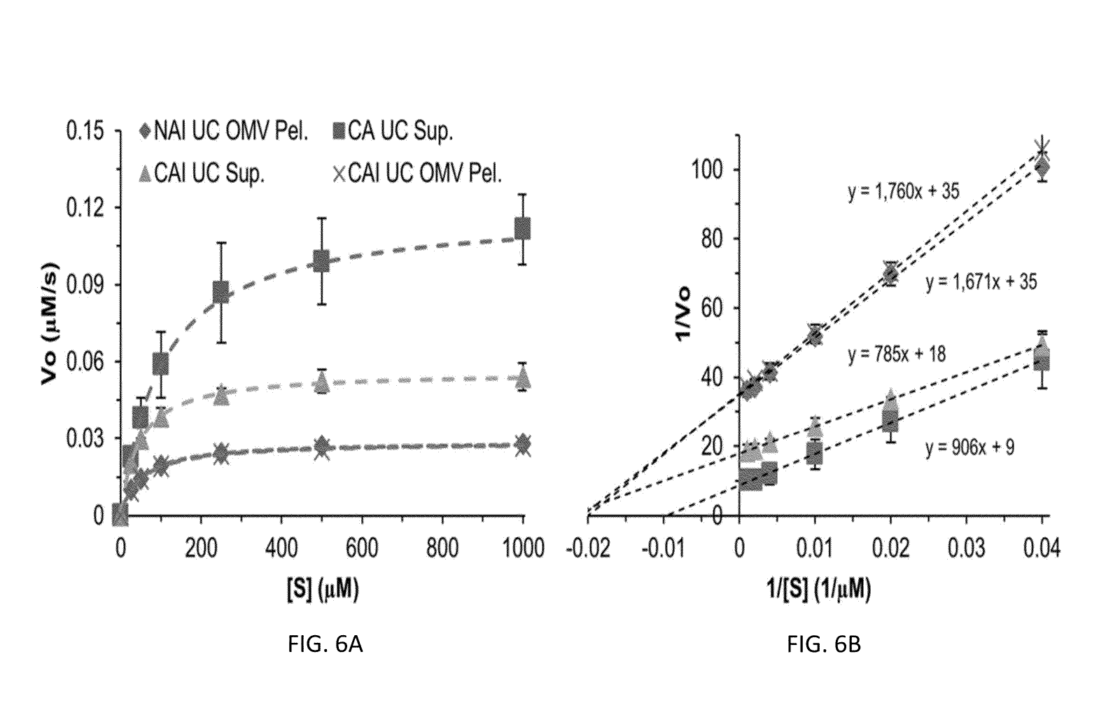

The enzyme kinetic characteristics for the free PTE-SC and the packaged PTE-SC were compared for some of the constructs of interest. The CA ultracentrifuged supernatant served as an internal control to represent free PTE-SC kinetic values. Utilizing a Lineweaver-Burk analysis all of the samples fit the traditional Michaelis-Menten enzyme kinetic model very well with R2>0.99 (FIGS. 6A and 6B). KM, Vmax, and kcat values were calculated for the CA free PTE-SC of 103.7 μM, 0.119 μM/s, and 2320 s-1, respectively. These values were consistent with the literature values of PTE isolated from B. diminuta with a Zn/Zn binuclear metal ion active site under similar assay conditions. (35) While the amount of free PTE-SC in solution was quantifiable, this was not feasible with regard to the total amount of active PTE-SC encapsulated within the OMV for each construct via absorbance or densitometry, necessary for the kcat determination, was not due to the expression levels observed and sample complexity. The kcat value of 2320 s-1, as determined from free PTE-SC, was therefore used to estimate the amount of PTE-SC encapsulated within the OMVs in the other samples based upon experimentally determined initial velocity values. This kcat was compared across samples since the initial velocity measurements for OMV encapsulated PTE-SC did not differ greatly when compared to the ruptured OMVs in the presence of Triton X-100. The remaining samples exhibited very similar KM, kcat, and kcat/KM values to one another averaging across all other samples tested 47.3±3.1 μM, 2088.7±47.8 s-1, and 4.42×107±0.23×107 (s-1 M-1), respectively. These values mirror accepted literature values for native PTE, KM=90 μM, kcat=2400 s-1, kcat/KM=2.7×107 (s-1 M-1), and further demonstrate that the OMVs do not inhibit the transfer of paraoxon through the vesicle membrane and that the PTE-SC produced is in a highly active native conformation.

Packaged PTE Freeze-Thaw Stability

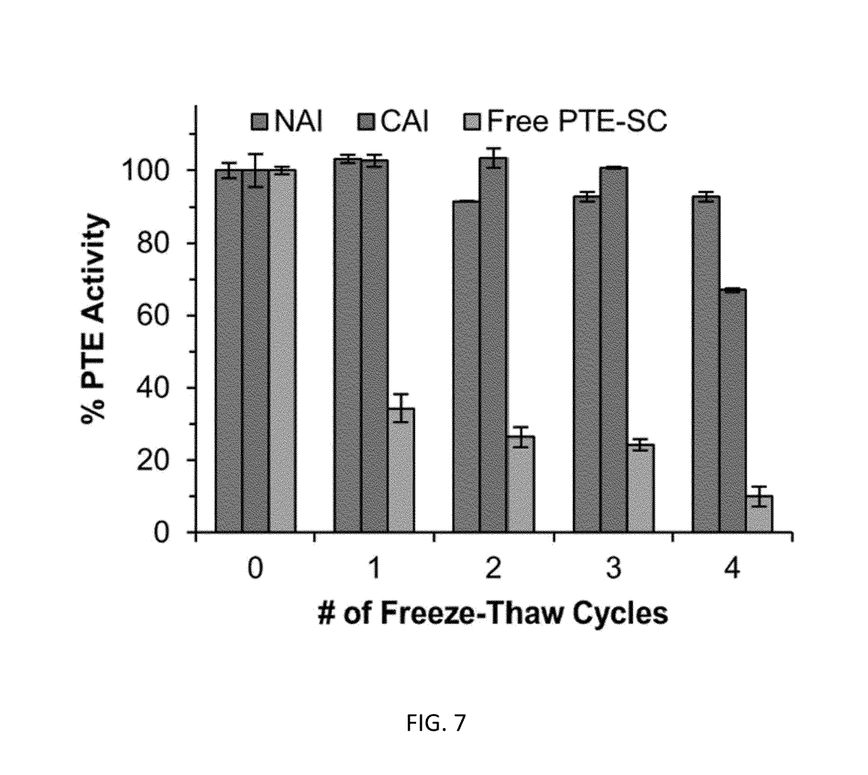

It was suspected that packaging the PTE-SC within OMVs would enhance the enzyme stability and therefore reduce endogenous inactivation of the enzyme. Enzymes are notoriously unstable and are susceptible to reduced activity even over very short periods of time. To explore this idea, the NAI and CAI packaged PTE-SC constructs were subjected to a series of freeze-thaw cycles and compared the percent PTE activity remaining after each cycle to free PTE-SC subjected to the same experimental conditions. Freeze-thaw is often considered to be one of the most detrimental conditions that a sensitive protein can be subjected to and was utilized here to definitively assess how OMV packaged PTE compared to free PTE. Free PTE-SC was purified from the UC supernatant of the CAI construct via immobilized metal ion affinity chromatography (IMAC) utilizing the included 6×Histag. Stock samples were aliquoted and exposed to four cycles of freeze-thaw between −80° C. and room temperature. PTE activity was directly compared to the initial PTE-SC activity of the same sample not subjected to any freeze-thaw cycles via comparison of the initial velocity measurements utilizing paraoxon as a substrate to calculate percent PTE activity remaining after each cycle. There was a marked decrease in the activity of free PTE-SC dropping to 34% after one cycle and exhibiting only 10% remaining activity after four cycles of freeze-thaw (FIG. 7). This is in comparison to the 93 and 67% remaining activity of packaged PTE-SC in the NAI and CAI constructs after four cycles of freeze-thaw, respectively. This demonstrates a 9.3- and 6.7-fold increase in active PTE-SC remaining after four freeze-thaw cycles in the NAI and CAI packaged constructs compared to naked PTE-SC. The NAI packaged PTE-SC demonstrated an increased resistance to inactivation from freeze-thaw compared to the CAI packaged PTE-SC. This result was expected since the CAI construct exhibited heightened membrane destabilization compared with the NAI construct in the aforementioned experiments which likely would provide less protection to the encapsulated enzyme compared to a fully intact membrane. Through packaging the PTE within OMVs the enzyme is much less susceptible to inactivation making this functional material a powerful and robust reagent compared to free enzyme allowing for improved implementation under harsh conditions

Discussion

This demonstrated a method for increasing vesiculation and improving the packaging efficiency of a periplasmically produced active enzyme, PTE-SC. The OMV packaged PTE-SC was capable of breaking down paraoxon that passively entered the vesicle through transmembrane porin proteins and exhibited kinetic parameters comparable to native, free PTE. OmpA was successfully employed as a membrane anchor to facilitate packaging of the target enzyme.

In addition to OMV packaging, it was observed that PTE-SC production was significantly increased when compared to traditional cytoplasmic and periplasmically targeted methods of protein production through the addition of the OmpA-ST mutant. While overall PTE-SC expression levels varied, there was an increase in PTE-SC production across all ST fusion locations tested. The increased PTE production correlates in most instances with increased OMV production. It is believed that both OMV packaging and reduction in membrane integrity contribute to PTE export from the cell and therefore a reduction in the toxicity allowing for production of higher concentrations of the recombinant enzyme.

While none of the SpyTag/SpyCatcher fusion constructs resulted in a complete conversion of free PTE-SC to covalently fused PTE-SC/OmpA-ST, it is evident that the presence of the ST and SC help drive packaging of the PTE into the OMVs. The location of the ST fusion was critical for the packaging efficiency of the PTE-SC, and we found that the C-terminal fusion of SpyTag to the mutant OmpA, in the presence or absence of IPTG activation, resulted in not only the highest total amount of PTE-SC produced but also the highest levels of PTE-SC packaged within the OMVs.

The packaged PTE-SC was much less susceptible to inactivation via exposure to multiple freeze-thaw cycles compared to free PTE-SC. This result is important as it demonstrates that the functional biological nanoparticles created by this method are far more robust allowing for implementation in more harsh environments providing for increased opportunities for use in applications that may not have been previously possible. The results of this study can be broadly applied to other outer membrane vesicle packaging systems across various applications to both increase vesiculation, drive enzyme packaging, and enhance enzymatic stability through OMV encapsulation. Though still in its infancy, careful design of bacterial synthesis pathways and the export of proteins, small molecules, and nucleic acids as OMV cargo will continue to be used to develop novel materials for environmental remediation, therapeutics, and methods of controlling the properties of microbial communities

Advantages and New Features

The above-described technique provides many advantages.

Sequestration of recombinant products in a proteoliposomes that provides protection from extracellular proteases.

Increased concentration of recombinant products within a confined space confers advantages such as: potential for enhanced catalytic activity of one or more encapsulated enzymes, and elevated local concentrations of recombinant products when OMVs serve as targeted therapeutic delivery vehicles.

The technique could allow for the simultaneous production and purification of multiple products from a single microbial culture.

Persistent removal of accumulating recombinant products during the production stage of growth (expression conditions) potentially alleviates adverse effects of cell toxicity that can be encountered due to properties of the recombinant product and thereby increase levels of production.

Improved stability under conditions of storage typically employed for recombinant products including but not limited to lyophilization, freeze-thaw cycles, extended refrigeration, and/or extended storage at ambient and elevated temperatures.

Production of the recombinant product in a vehicle (proteoliposomes) that is suitable for storage over a range of conditions or to serve as a delivery vehicle for environmental or therapeutic applications with the potential for recombinant and post production surface modification (such as external proteolytic digestion to remove outer proteins) to endow the vehicle with active targeting or stealth capabilities.

The technique enable producing and loading vesicle structures from a single microbial culture eliminating complex processes of iterative loading, synthesis, and purification that would be required to reproduce this process employing synthetic liposomes/vesicles using in vitro techniques

Modifications can be made to the OMVs to expand the potential applications of the filled OMVs. Exemplary, non-limiting modifications to the exterior face of OMVs include the following. For instance, a targeting moiety can cause OMVs to target either prokaryotic or eukaryotic cells. The targeting moiety can be a recombinant antibody or functional fragment thereof; a cell penetrating peptide; and/or one or more receptors or ligands complementary to features of the target cell through an in vitro method such as click chemistry of carbodiimide-mediated attachment. It is also possible to include reactive chemical group to facilitate modification of the OMV surface. One could arrange for the presentation of a functional enzyme of protein on the exterior surface of the OMV. It may be desirable to remove or modify any lipopolysaccharide present in or on the OMV and/or remove or modify the antigenicity of the OMV.

The aforementioned processes of external modification and lumenal packaging can be combined to generate tools and reagents for a range of applications including but not limited to: incorporation of fluorescent proteins to create optical probes in conjunction with incorporation or chemical modification to a targeting entity such as an antibody. As multiple copies of the fluorescent protein will be incorporated and potentially stabilized in the OMV—the probes should be capable of resisting photo-, chemical and proteolytic degradation to some extent. Potential exists to use DNA-binding proteins in the OMV to allow packaging of nucleic acid cargos. These could be used to regulate or modify cellular functions, pathways, or even induce cytotoxic effects.

Concluding Remarks

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

1. A method of producing a protein, the method comprising:
providing a bacterial cell expressing both (a) a protein of interest fused to one of the SpyTag/SpyCatcher pair and (b) an outer membrane protein fused to the other of the SpyTag/SpyCatcher pair;
causing the bacterial cell to express both of the protein of interest fusion and the outer membrane protein fusion in outer membrane vesicles; and
purifying the outer membrane vesicles.

2. The method of claim 1, wherein the protein of interest is an enzyme.

3. The method of claim 2, wherein the enzyme is phosphotriesterase.

4. The method of claim 1, wherein the outer membrane protein is OmpA.

5. The method of claim 1, wherein
the protein of interest fusion and the outer membrane protein fusion are each under the control of different inducible promoters, and
further comprising separately inducing the expression of each of the protein of interest and the outer membrane protein.

6. The method of claim 1, wherein said purifying comprises affinity purification.

7. A method of producing a protein, the method comprising:
providing a bacterial cell expressing both (a) a protein of interest fused to a first member of a conjugation pair and (b) an outer membrane protein fused to a second member of the conjugation pair;
causing the bacterial cell to express both of the protein of interest fusion and the outer membrane protein fusion in outer membrane vesicles; and
purifying the outer membrane vesicles.


SEQUENCE LISTING (10 sequences)

SEQ ID NO: 1 - protein, 218 aa, Artificial Sequence
  synthetic construct
MKKTAIAIAVALAGFATVAQAGGGSAHIVMVDAYKPTKGGGSAPKDNTWYTGAKLGWSQYHDTGFINNNGPTHENQLGAGAFGGYQVNPYVGFEMGYDWLGRMPYKGSHHHVENGAYKAQGVQLTAKLGYPITASDDLDIYTRLGGMVWRADTKSNVYGKNHDTGVSPVFAGGVEYAITPEIATRLEYQWTNNIGDAHTIGTRPDNGMLSLGVSYRFG

SEQ ID NO: 2 - protein, 216 aa, Artificial Sequence
  synthetic construct
MKKTAIAIAVALAGFATVAQAAPKDNTWYTGAKLGWSQYHDTGFINNNGPTHENQLGAGAFGGYQVNPYVGFEMGYDWLGRMPYKGSHHHVENGAYKAQGVQLTAKLGYPITGGGSAHIVMVDAYKPTKGGGSDDLDIYTRLGGMVWRADTKSNVYGKNHDTGVSPVFAGGVEYAITPEIATRLEYQWTNNIGDAHTIGTRPDNGMLSLGVSYRFG

SEQ ID NO: 3 - protein, 60 aa, Artificial Sequence
  synthetic construct
MKKTAIAIAVALAGFATVAQAAPKDNTWYTGAKLGWSQYHDTGFINNNGPTHENQLGAGA

SEQ ID NO: 4 - protein, 518 aa, Artificial Sequence
  synthetic construct
MNNNDLFQASRRRFLAQLGGLTVAGMLGPSLLTPRRATAAQARGSHHHHHHLEGTSIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDRVNPDGMAFIPLRVIPFLREKGVPQETLAGITVTNPARFLSPTLRASGTGGSVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHISGGGGELVD

SEQ ID NO: 5 - protein, 21 aa, Artificial Sequence
  synthetic construct
MKKTAIAIAVALAGFATVAQA

SEQ ID NO: 6 - protein, 13 aa, Artificial Sequence
  synthetic construct
AHIVMVDAYKPTK

SEQ ID NO: 7 - protein, 176 aa, Artificial Sequence
  synthetic construct
APKDNTWYTGAKLGWSQYHDTGFINNNGPTHENQLGAGAFGGYQVNPYVGFEMGYDWLGRMPYKGSHHHVENGAYKAQGVQLTAKLGYPITASDDLDIYTRLGGMVWRADTKSNVYGKNHDTGVSPVFAGGVEYAITPEIATRLEYQWTNNIGDAHTIGTRPDNGMLSLGVSYRFG

SEQ ID NO: 8 - protein, 45 aa, Artificial Sequence
  synthetic construct
MNNNDLFQASRRRFLAQLGGLTVAGMLGPSLLTPRRATAAQARGS

SEQ ID NO: 9 - protein, 113 aa, Artificial Sequence
  synthetic construct
VDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 10 - protein, 337 aa, Brevundimonas diminuta
SIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDRVNPDGMAFIPLRVIPFLREKGVPQETLAGITVTNPARFLSPTLRASG